(19) United States
(12) Patent Application Publication
(10) Pub. No.: US 2004/0191872 A1
(43) Pub. Date: Sep. 30, 2004

(54) Process for preparation of RNA using affinity column and DNAzyme

(75) Inventors: Chaejoon Cheong, Daejeon-city (KR); Chulhyun Lee, Daejeon-city (KR); Hae-Kap Cheong, Daejeon-city (KR); Jun-Soek Yoo, Daejeon-city (KR); Eunha Hwang, Daejeon-city (KR)

Correspondence Address: STAAS & HALSEY LLP, SUITE 700, 1201 NEW YORK AVENUE, N.W., WASHINGTON, DC, 20005, US

(73) Assignee: Korea Basic Science Institute, Daejeon-city (KR)

(21) Appl. No.: 10/438,992

(22) Filed: May 16, 2003

(30) Foreign Application Priority Data
Mar. 28, 2003 (KR) 2003-0019636

(51) Int. Cl. C12P 19/34; C07H 21/02

(52) U.S. Cl. 435/91.2; 536/23.1

(57) ABSTRACT

A process for preparation of RNA using an affinity column and a DNAzyme is disclosed. By the process, a target RNA can be mass-produced with a high yield, a high resolution and a high purification efficiency. The process includes the steps of preparing a RNA adduct including a target RNA and a tail RNA sequence which is affixed to the 3′-terminal of the target RNA and can complementary combine with an oligo-dN sequence of an oligo-dN affinity column; purifying the RNA adduct by combining the RNA adduct to the oligo-dN affinity column; and obtaining the target RNA by cleaving the purified RNA adduct with a DNAzyme. Preferably, the process for preparation of RNA further includes the steps of removing a RNA fragment cleaved by the DNAzyme by using an affinity column; and decomposing the DNAzyme with a DNA nuclease, and then removing decomposed DNAzyme fragments with a gel filtration column.

### FIELD OF THE INVENTION

The present invention relates to a process for preparation of RNA, and more particularly, to a process for preparation of RNA using an affinity column and a DNAzyme, in which a target RNA can be mass-produced with a high yield, a high resolution and a high purification efficiency.

### BACKGROUNDS OF THE INVENTION

Along with the entrance into the post-genome era, functional genomics for researching the functions of proteins and RNAs, which are final products of genes, have attracted much attentions world-widely. Especially, the structural genomics, which investigate the three-dimensional structures of biological polymers to clarify the functions thereof in a rapid and accurate manner, has lately attracted considerable attention. In the structural genomics utilizing X-ray crystallography or NMR for researching the three-dimensional structures of the biological polymers, a large amount of purified sample is required, and such RNA sample is conventionally prepared by a preparative polyacrylamide gel electrophoresis (PAGE). The preparative PAGE is known as an only practical method for purifying RNA products synthesized by in vitro transcription with nucleotide resolution. However, the method has an important disadvantage that it requires much time and much amount of work to be done. By this reason, the process is regarded as “a rate-determining step” of RNA structural research. Contrary to the simple column chromatography, even though a system for carrying out the electrophoresis for several plates at the same time is used, one person can perform the electrophoresis for at most 4 plates a day in the preparative PAGE method. Furthermore, in the preparative PAGE, the amount of sample which can be loaded to the acrylamide gel is limited to a predetermined amount (about 50 OD for a plate), and the same electrophoresis purification step should be carried out repeatedly for a sample of more than the predetermined amount. If the number of sample is small, this would not induce a serious problem, but if the purification step is carried out for the large amount and several samples, this would induce a serious problem. Meanwhile, in the in vitro transcription, n±1 RNA transcript, which has the nucleotide number one less or one more than the nucleotide number of the target RNA sequence, is liable to be produced. If the mobility of the n±1 RNA transcript is similar to that of the target RNA, the resolution of the purification should be increased by reducing the amount of the sample loaded to the gel, which requires more time for the purification. As another disadvantages of using the preparative PAGE, the loss of RNA sample could occur during the purification process, and the residual n±1 RNA transcript and acrylamide impurities which can be exist in the purified RNA sample may induce an adverse effect in the next analysis step, such as NMR analysis.

Recently, the information on the genes for encoding proteins and functional RNAs increases enormously. Thus, it is required a high-throughput process (HTP) for preparation of RNA which can produce RNA sample with high efficiency, and which makes possible to conduct a research on the various genes in the industrial large scale.

### SUMMARY OF THE INVENTION

Therefore, the present invention is to provide a process for preparation of RNA which is capable of mass-producing target RNA with a high yield, a high resolution, and high purification efficiency. Other object of the present invention is to provide a process for preparation of RNA, which does not require much time and much amount of work to be done, and which is economically advantageous.

To achieve these objects, the present invention provides a process for preparation of RNA comprising the steps of preparing a RNA adduct including a target RNA and a tail RNA sequence which is affixed to the 3′-terminal of the target RNA and can complementary combine with an oligo-dN sequence of an oligo-dN affinity column; purifying the RNA adduct by combining the RNA adduct to the oligo-dN affinity column; and obtaining the target RNA by cleaving the purified RNA adduct with a DNAzyme. Preferably, the process for preparation of RNA further includes the steps of removing a RNA fragment cleaved by the DNAzyme by using an affinity column; and decomposing the DNAzyme with a DNA nuclease, and then removing decomposed DNAzyme fragments with a gel filtration column.

### BRIEF DESCRIPTION OF THE DRAWINGS

FIG. 1 is a drawing for illustrating the RNA cleaving function of a DNAzyme.

FIG. 2 is a drawing for illustrating the step of cleaving the head and the tail RNA sequences affixed to a target RNA with two different DNAzyme according to an embodiment of the present invention.

FIG. 3 is a photograph showing the PAGE results of a RNA adduct including a target RNA and a head and/or a tail RNA sequences which are affixed to the 3′-terminal and/or 5′-terminal of the target RNA according to an embodiment of the present invention.

FIG. 4 is a photograph showing the PAGE results of a RNA adduct purified by an affinity column according to an embodiment of the present invention.

FIG. 5 is a photograph showing the PAGE results of RNA fragments cleaved with a DNAzyme according to an embodiment of the present invention.

FIG. 6 is a photograph showing the PAGE results of a target RNA fragment after and before removing a head and/or a tail RNA fragments with an affinity column according to an embodiment of the present invention.

### DETAILED DESCRIPTION OF THE INVENTION

For the better understanding of the present invention, reference will now be made in detail to the following disclosures and appended drawings.

In order to prepare RNA with a high efficiency according to the present invention, a RNA adduct including a target RNA and a tail RNA sequence is synthesized. The tail RNA sequence is affixed to the 3′-terminal of the target RNA and is designed to complementary combine with an oligo-dN (oligo deoxynucleotides) sequence of an oligo-dN affinity column. If necessary, the RNA adduct further includes a head RNA sequence which is affixed to the 5′-terminal of the target RNA, and the head RNA sequence is designed to increase the transcription yield of the RNA adduct. The RNA adduct can be synthesized by conventional RNA synthesizing method, and for example, can be synthesized by in vitro transcription using a T7 RNA polymerase and a chemically produced DNA template. By affixing the tail RNA sequence to the 3′-terminal of the target RNA, the smearing phenomena of the RNA band, which may occur when the conventional poly A₂₀ is affixed to the target RNA, can be prevented. The tail RNA sequence affixed to the 3′-terminal of the target RNA is determined according to the oligo-dN sequence of the oligo-dN affinity column, and for example, may have the nucleotide sequence of MGMGCCCUUCAG and so on. The head RNA sequence affixed to the 5′-terminal of the target RNA is experimentally designed in consideration of the properties of the T7 RNA polymerase so that the transcription yield can be improved, and preferably, may have the nucleotide sequence starting with G or GG, such as GGGAGA, GGAGACGC and so on.

Then the synthesized RNA adduct is purified with the oligo-dN affinity column. The oligo-dN affinity column used in the present invention can be prepared by synthesizing oligo-dN on the surfaces of resin particles of a column, such as polystyrene, controlled pore glass(CPG), cellulose and so on. The specific example of the purification process of the RNA adduct with the oligo-dN affinity column will be described hereinafter. The synthesized RNA product including the RNA adduct is introduced into the oligo-dN affinity column to anneal the RNA adduct to the oligo-dN affinity column in the presence of a binding buffer solution, such as, 250 mM NaCl, 20 mM sodium phosphate buffer solution (pH 7.0). Then, the column, on which the RNA adduct is annealed, is washed several times with a washing buffer, such as, with 250 mM NaCl, 20 mM sodium phosphate solution (pH 7.0). Thereafter, an elution buffer, such as 1 mM sodium phosphate solution (pH 7.0) is added to the column to obtain the purified RNA adduct from the column.

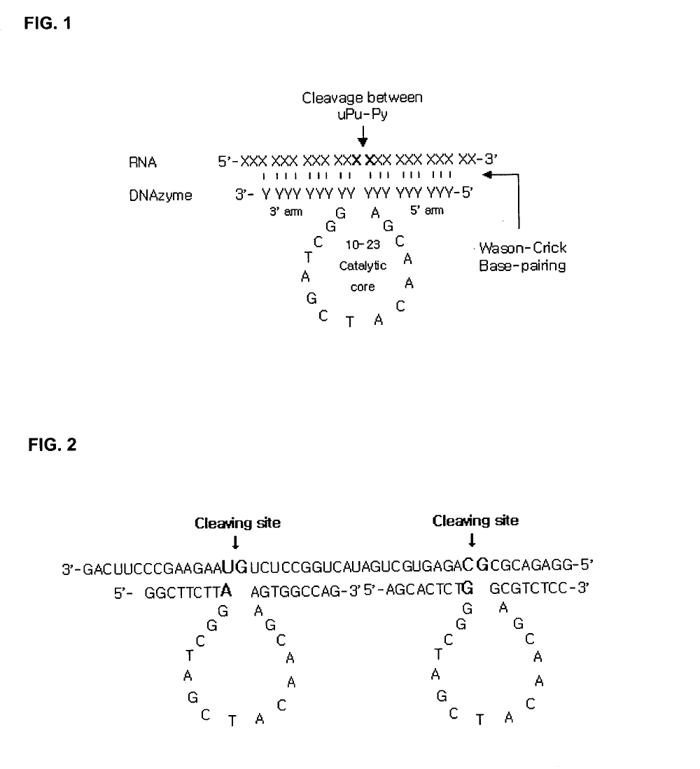

The purified RNA adduct is cleaved with a DNAzyme to produce the target RNA. FIG. 1 is a drawing for illustrating the RNA cleaving function of a DNAzyme. As shown in FIG. 1, an exemplary DNAzyme is a RNA-cleaving deoxyribozyme which forms Watson-Crick base-pairing with 7-8 nucleotides for each side at the head and tail sides of an RNA, and includes 15 nucleotide sequence of 5′-GGCTAGCTACAACGA-3′ as a Mg²⁺-dependent catalytic domain, and cleaves the phosphodiester linkage between the unpaired purine(A, G) and the paired pyrimidine (C, U) of the RNA. When predetermined RNA sequences are affixed to the 5′-terminal and 3′-terminal of the target RNA for the RNA purification and for increasing the yield of RNA synthesis, the two affixed sequences, i.e., the head and the tail RNA sequences can be cleaved with two different DNAzymes, as shown in FIG. 2. The DNAzyme is designed to cleave the affixed RNA sequences according to the sequence of the RNA adduct. If necessary, before cleaving the affixed sequence with the DNAzyme, the impurities, such as salts, in the purified RNA adduct can be removed with a desalting column. When the two different DNAzymes are used, they can be used at the same time or sequentially, and the RNA cleaving reaction is preferably carried out after incubating the reaction solution such as with 40 mM Tris-HCl (pH 7.5), 150 mM NaCl and 60 mM MgCl₂.

After splitting the RNA adduct to the target RNA and the head and/or tail RNA fragments, the head and the tail RNA fragments are preferably removed from the DNAzyme reaction product by using an affinity column, which adsorbs the cleaved head and tail RNA fragments. Then, the DNAzyme is preferably decomposed with a DNA nuclease, such as DNase I, and the decomposed DNAzyme fragments are removed by using a gel filtration column, such as Superdex 75 HR to obtain the final target RNA.

Hereinafter, the preferable example is provided for better understanding of the present invention. However, the present invention should not be restricted to the following Example.

A. Synthesis of RNA The core element having the following structure was selected from the 3′-noncoding region (3′-NCR) of a C-type hepatitis virus(HCV I) for RNA synthesis. The core element includes about 100 nucleotides, and has a two dimensional structure which is maintained in most genotypes. Among the core element, SLI domain RNA was synthesized as follows.

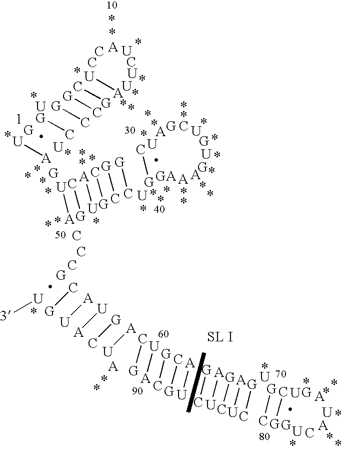

RNA adducts were designed so that the nucleotide sequence of GGAGACGC was affixed to the 5′-termianl of the SLI domain to increase the transcription yield of the RNA synthesis, and the nucleotide sequence of AAGMGCCCUUCAG, instead of poly A₂₀, was affixed to the 3′-termianl of the SLI domain. By using a T7 RNA polymerase and a chemically produced DNA template, the RNA adduct having the following nucleotide sequence 1 was synthesized, and the RNA adduct having the following nucleotide sequence 2 without the nucleotide sequence of GGAGACGC affixed to the 5′-termianl was synthesized.

[Nucleotide sequence 1]
5′-
GGAGACGCGCAGAGUGCUGAUACUGGCCUCUGUAAGAAGCCCUUCAG-3′
[Nucleotide sequence 2]
5′-GAGAGUGCUGAUACUGGCCUCUGUAAGAAGCCCUUCAG-3′

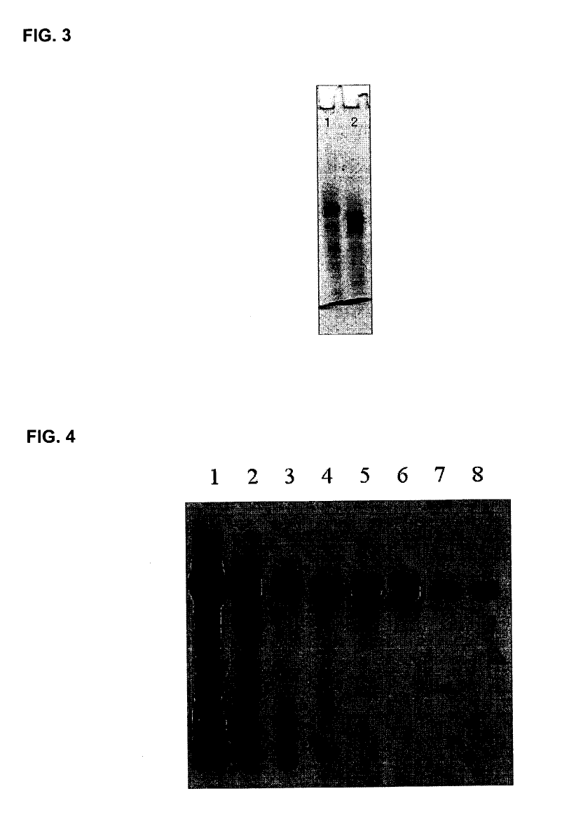

FIG. 3 is a photograph showing the PAGE results of the produced RNA adducts. In FIG. 3, the bands of the first lane represent 47-mer RNA of the nucleotide sequence 1, and the bands of the second lane represent 38-mer RNA of the nucleotide sequence 2. As shown in FIG. 3, by designing the RNA adduct so that the nucleotide sequence of GGAGACGC is affixed to the 5′-termianl of the SLI domain, and the nucleotide sequence of AAGAAGCCCUUCAG, instead of poly A₂₀, is affixed to the 3′-termianl of the SLI domain, the transcription yields of the RNA adducts increases, and the smearing phenomena of the RNA band is prevented.

B. Preparation of Affinity Column and DNAzyme

An oligo-dN affinity column was prepared by synthesizing oligo-dN on the surfaces of polystyrene-latex with an automated nucleotide synthesizer. As the DNAzyme, 32-mer DNA having the following nucleotide sequence 3 and 33-mer DNA having the following nucleotide sequence 4 were prepared.

[Nucleotide sequence 3]
5′-d(AGCACTCTGGGCTAGCTACAACGAGCGTCTCC)-3′
[Nucleotide sequence 4]
5′-d(GGCTTCTTAGGCTAGCTACAACGAAGTGGCCAG)-3′

C. Purification of RNA with Affinity Column

The obtained oligo-dN affinity column was mixed with a binding buffer (250 mM NaCl, 20 mM sodium phosphate buffer, pH 7.0), and the synthesized RNA adduct was introduced thereto. The mixture was allowed at 70° C. for 5 minutes, and then slowly cooled to anneal the RNA adduct to the column. After annealing the RNA adduct to the column, un-adsorbed materials were eluted with same buffer, and the column was washed two times with a washing buffer (250 mM NaCl, 20 mM sodium phosphate, pH 7.0). Thereafter, an elution buffer (1 mM sodium phosphate, pH 7.0) is added to the column, allowed at 70° C. for 5 minutes, and the purified RNA adduct was obtained by elution from the column.

FIG. 4 is a photograph showing the PAGE results of the RNA adduct purified by the affinity column. In FIG. 4, the bands of the first lane represent the bands of the synthesized RNA transcript before purification with the affinity column; the bands of the second and the third lanes represent RNAs in the affinity column washing buffer; and the bands of the fourth to eighth lanes represent RNAs in the eluate eluted with the elution buffer. As shown in FIG. 4, the abortive transcripts, N−1 or N+1 mer produced in the RNA synthesis is removed during the purification with the affinity column. In the PAGE shown in FIG. 4, the electrophoresis was carried out with 15% polyacrylamide −7M urea denaturing gel, and the detection was performed with toluidine blue.

D. Cleavage of RNA with DNAzyme

The impurities, such as salts, in the obtained supernatant were removed with a desalting column, and the two synthesized DNAzymes in 0.1×DNAzyme reaction buffer (15 mM NaCl, 4 mM Tris-HCl, pH 8.0) were added thereto. The mixture was successively allowed to spin-down, heated at 95° C. for 3 to 4 minutes, allowed at ice bath for 5 minutes, and incubated at 25° C. for 10 minutes. Then, 5×DNAzyme reaction buffer (750 mM NaCl, 200 mM Tris-HCl, pH 8.0) was added thereto so that the concentration became 1×, i.e., the buffer was diluted by 5 times. The mixture was annealed at 37° C. for 1 hour, and 60 mM MgCl₂ was added thereto, and then the reaction was allowed at 37° C. for 4 hours. After the reaction was completed, the RNA cleavage was observed by carrying out the 15% polyacrylamide −7M urea denaturing gel electrophoresis for the reaction product of the DNAzymes and the RNA adduct.

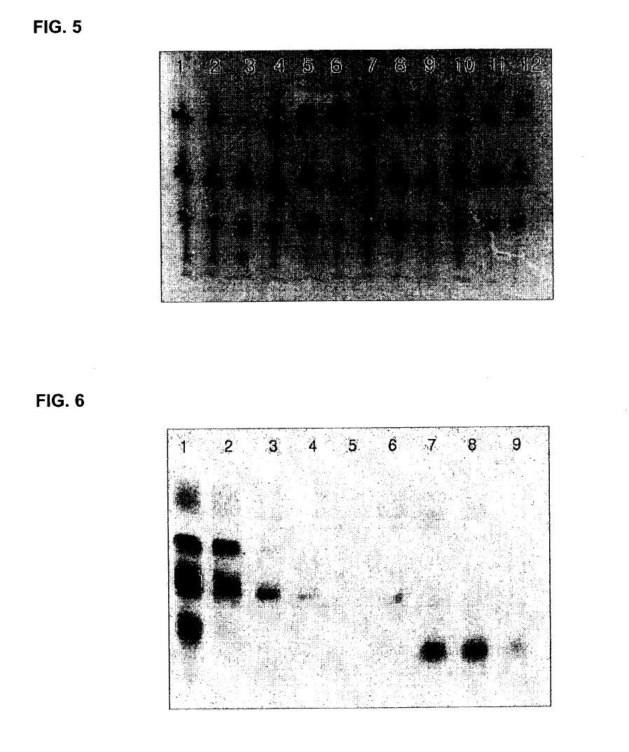

FIG. 5 is a photograph showing the PAGE results of the RNA fragments cleaved with the DNAzymes. In FIG. 5, the bands of the first to third lanes represent the PAGE results after incubating the reaction product overnight; the bands of the fourth to sixth lanes represent the PAGE results after incubating the reaction product for 1 hour; the bands of the seventh to ninth lanes represent the PAGE results after incubating the reaction product for 2 hours; and the bands of the tenth to 12^{th} lanes represent the PAGE results after incubating the reaction product for 3 hours. In the first to ₁₂^{th} lanes, the uppermost band is the DNAzyme band, the middle band is the target RNA band synthesized for clarifying the structure, and the lowermost bands are the RNA fragment bands, i.e., the head and tail RNA sequences which are affixed for RNA purification and for increasing the transcription yield. From FIG. 5, it is confirmed that the tail RNA sequence which was affixed to the target RNA for RNA purification, and the target RNA were split by the DNAzyme.

In the next step, the head and/or tail RNA fragments were removed from the DNAzyme reaction product by using an affinity column, which adsorbs the cleaved head and tail RNA fragments. FIG. 6 is a photograph showing the PAGE results of the target RNA fragment after and before removing the head and/or the tail RNA fragments with the affinity column. In FIG. 6, the bands of the first lane represent the bands of the DNAzyme reaction product; the bands of the second lane represent the bands of the DNAzyme and the target RNA purified with the affinity column; the bands of the third and fourth lanes represent the bands of RNA in the washing buffer of the affinity column; the bands of the fifth to ninth lanes represent the bands of RNA in the eluate eluted with an elution buffer of the affinity column. Finally, in order to remove the DNAzyme, the DNAzyme was treated with DNase I, which is one DNA nucleases, and the produced dNTP and the RNA were separated with size exclusion column to obtain the final target RNA.

As previously described, the high-throughput process for preparation of RNA using affinity column and DNAzyme according to the present invention is useful in mass-producing RNA with a high yield, and requires less time and less work to be done compared to the conventional PAGE method. In the process of the present invention, the sequence homology at the 3′-terminal of the RNA adduct can be attained, and thereby the purification can be carried out with nucleotide resolution. In addition, by using a head RNA sequence affixed to the 5′-terminal of the target RNA, experimental restrictions due to the starting sequence can be prevented, and the transcription yield can be improved. While the present invention has been described with respect to certain preferred embodiments and examples only, other modifications and variations may be made without departing from the spirit and scope of the present invention as set forth in the following claims.

[References]

1. Alessio Peracchi, The Journal -of Biological Chemistry, 275, 11693 (2000)

2. Christopher W. J. Smith, RNA: Protein Interaction, 38 (1998)

3. Daniel W. Celander and John N. Abelson, Methods in Enzymology, 317, 140 (2000)

4. Gow-Yuan Lee, Chi-Hao Chen, Tzu-Hsien Wang, and Wen-Chien Lee, Analytical biochemistry, 312, 235 (2003)

5. Levon M. Khachigian, The Journal of Clinical Investigation, 106, 1189 (2000)

6. L. Q. Sun, M. J. Cairns, E. G. Saravolac, A. Baker, W. L. Gerlach, Pharmacological Reviews, 52, 325 (2000)

7. Milan N. Stojanovic, Paloma de Prada and Donald W. Landry, Nucleic Acids Research, 28, 2915 (2000)

8. Mouldy Sioud and Marianne Leirdal, Biochemical Pharmacology, 60, 1023 (2000)

9. Naruhisa Ota, Masaki Warashina, Ken'ichi Hirano, Kenichi Hatanaka and Kazunari Taira, Nucleic Acids Research, 26, 3385 (1998)

10. Ronald R Breaker and Gerald F Joyce, Chemistry & Biology, 2, 655 (1995)

11. Stephen W. Santoro and Gerald F. Joyce, Proc. Natl. Acad. Sci. USA, 94, 4262 (1997)

12. Stephen W. Santoro and Gerald F. Joyce, Biochemistry, 37, 13330 (1998)

What is claimed is:

1. A process for preparation of RNA comprising the steps of:
preparing a RNA adduct including a target RNA and a tail RNA sequence which is affixed to the 3′-terminal of the target RNA and can complementary combine with an oligo-dN sequence of an oligo-dN affinity column;
purifying the RNA adduct by combining the RNA adduct to the oligo-dN affinity column; and
obtaining the target RNA by cleaving the purified RNA adduct with a DNAzyme.

2. The process for preparation of RNA according to claim 1, further comprising the steps of:
removing a RNA fragment cleaved by the DNAzyme by using an affinity column; and
decomposing the DNAzyme with a DNA nuclease, and then removing decomposed DNAzyme fragments with a gel filtration column.

3. The process for preparation of RNA according to claim 1, wherein the RNA adduct is synthesized by using a T7 RNA polymerase and a chemically produced DNA template.

4. The process for preparation of RNA according to claim 1, wherein the RNA adduct further includes a head RNA sequence which is affixed to the 5′-terminal of the target RNA to increase a transcription yield.

5. The process for preparation of RNA according to claim 1, wherein the oligo-dN affinity column is prepared by synthesizing oligo-dN on the surfaces of resin particles selected from the group consisting of polystyrene, controlled pore glass(CPG), cellulose and the mixtures thereof.

6. The process for preparation of RNA according to claim 1, wherein the tail RNA sequence which can complementary combine with the oligo-dN sequence of the oligo-dN affinity column has the nucleotide sequence of AAGAAGCCCUUCAG.

7. The process for preparation of RNA according to claim 1, wherein the DNAzyme cleaves a phosphodiester linkage between an unpaired purine and a paired pyrimidine.


SEQUENCE LISTING (4 sequences)

SEQ ID NO: 1 - RNA, 47 nt, Artificial
  modified SLI domain of C-type hepatitis virus RNA
ggagacgcgcagagugcugauacuggccucuguaagaagcccuucag

SEQ ID NO: 2 - RNA, 38 nt, Artificial
  modified SLI domain of C-type hepatitis virus RNA
gagagugcugauacuggccucuguaagaagcccuucag

SEQ ID NO: 3 - DNA, 32 bp, Artificial
  DNAzyme
agcactctgggctagctacaacgagcgtctcc

SEQ ID NO: 4 - DNA, 33 bp, Artificial
  DNAzyme
ggcttcttaggctagctacaacgaagtggccag